(19) United States
(12) Patent Application Publication
(10) Pub. No.: US 2025/0157594 A1
(43) Pub. Date: May 15, 2025

(54) METHOD AND APPARATUS FOR DESIGNING LIGAND MOLECULES

(71) Applicants: Beijing Youzhuju Network Technology Co., Ltd., Beijing (CN); Lemon Inc., Grand Cayman (KY)

(72) Inventors: Yuwei Yang, Los Angeles, CA (US); Jiarui Lu, Beijing (CN); Shuo Zhang, Los Angeles, CA (US); Hao Zhou, Beijing (CN)

(21) Appl. No.: 18/839,008

(22) PCT Filed: Feb. 8, 2023
(86) PCT No.: PCT/CN2023/075067

§ 371 Date: Aug. 15, 2024

(30) Foreign Application Priority Data
Feb. 18, 2022 (CN) 202210152512.4

(51) Int. Cl. G16C 20/50; G16B 15/30; G16C 20/40; G16C 20/70

(52) U.S. Cl. CPC G16C 20/50; G16B 15/30; G16C 20/40; G16C 20/70

(57) ABSTRACT

According to embodiments of the present disclosure, a method, apparatus, device, storage medium, and program product for designing ligand molecules are provided. The method described herein comprises: editing a first 2D molecular structure to determine a second 2D molecular structure, the editing at least comprising: deleting a 2D structural fragment from the first 2D molecular structure, or adding a 2D structural fragment to the first 2D molecular structure; determining a set of candidate 3D molecular structures corresponding to the second 2D molecular structure based on a first 3D molecular structure corresponding to the first 2D molecular structure and the editing; determining a second 3D molecular structure corresponding to the second 2D molecular structure based on a binding capacity between the set of candidate 3D molecular structures and a target molecule; and determining a target structure of a ligand molecule for the target molecule based on the second 3D molecular structure. According to embodiments of the present disclosure, the generation of subsequent 3D molecular structures can be constrained based on the 3D molecular structure of the previous state, thereby improving the efficiency of designing ligand molecules.

### CROSS REFERENCE

The present application claims priority to the Chinese Patent Application No. 202210152512.4, filed on Feb. 18, 2022, entitled “Method and apparatus for designing ligand molecules”, which is incorporated herein by reference in its entirety.

### FIELD

Various implementations of the present disclosure relate to the field of computers, and more particularly, to methods, devices, devices, and computer storage media for designing ligand molecules.

### BACKGROUND

In drug discovery, an important task is to find drug small molecules (also known as ligand molecules, Ligand) that can effectively bind to target molecules (such as targeted protein molecules). In recent years, with the development of computer technology, computer-aided technologies such as Machine Learning have gradually been applied to the process of drug molecule discovery.

In the process of designing ligand molecules, it is usually necessary to consider the three-dimensional (3D) structure of the ligand molecule and the binding capacity between the target molecules. How to efficiently construct the 3D molecular structure is an important challenge in designing ligand molecules.

### SUMMARY

In a first aspect of the present disclosure, a method for designing ligand molecules is provided. The method comprises: editing a first 2D molecular structure to determine a second 2D molecular structure, the editing at least comprising: deleting a 2D structural fragment from the first 2D molecular structure, or adding a 2D structural fragment to the first 2D molecular structure; determining a set of candidate 3D molecular structures corresponding to the second 2D molecular structure based on a first 3D molecular structure corresponding to the first 2D molecular structure and the editing; determining a second 3D molecular structure corresponding to the second 2D molecular structure based on binding capacity between the set of candidate 3D molecular structure and a target molecule; and determining a target structure of a ligand molecule for the target molecule based on the second 3D molecular structure.

In some embodiments, editing a first 2D molecular structure comprising: determining an edit operation to be applied to the first 2D molecular structure with an operation prediction model and based on a feature representation corresponding to the first 2D molecular structure; and editing the first 2D molecular structure based on the determined edit operation.

In some embodiments, determining the edit operation to be applied to the first 2D molecular structure comprises: determining a set of probabilities associated with a set of predetermined edit operations with the operation prediction model and based on the feature representation, wherein the set of predetermined edit operations comprise: adding a specific 2D structure fragment at a specific atom in the first 2D molecular structure, or deleting a specific bond from the first 2D molecular structure; and determining the edit operation to be applied to the first 2D molecular structure from the set of predetermined edit operations based on the set of probabilities.

In some embodiments, adding a 2D structure fragment comprises: selecting a target 2D structure fragment from a fragment library, the fragment library including a plurality of 2D structure fragments; and adding the target 2D structure fragment to a specific atom in the first 2D molecular structure.

In some embodiments, determining a set of candidate 3D molecular structures corresponding to the second 2D molecular structure comprises: determining the set of candidate 3D molecular structures based on the editing with the first 3D molecular structure, wherein the set of candidate structures have a partial 3D structure corresponding to the first 3D molecular structure, the partial 3D structure corresponding to a partial 2D structure that is unmodified by the editing.

In some embodiments, the editing is adding a target 2D structure fragment to the first 2D molecular structure, and determining the set of candidate 3D molecular structures comprises: determining a configuration constraint based on the first 3D molecular structure corresponding to the first 2D molecular structure; generating a plurality of candidate 3D molecular structures corresponding to the editing based on the configuration constraint, configuration constraint specifying an extent to which the first 3D molecular structure is adjusted in process of generating the plurality of candidate 3D molecular structures; and performing an energy optimization on the plurality of candidate 3D molecular structures based on the configuration constraint to determine the set of candidate 3D molecular structures.

In some embodiments, the binding is determined based on a binding free energy between the set of candidate 3D structure fragments and the target molecule.

In some embodiments, determining the target structure of a ligand molecule for the target molecule comprises: determining a first evaluation for the second 3D molecular structure, the first evaluation indicating at least one of the following: a target binding capacity between the second 3D molecular structure and the target molecule, quantitative estimate of drug-likeness (QED) of the second 3D molecular structure, or synthesizability of the second 3D molecular structure; determining a probability of acceptance of the second 2D molecular structure based on the first evaluation and a second evaluation of the first 3D molecular structure; and determining the target structure based on the second 2D molecular structure and the second 3D molecular structure according to the probability.

In some embodiments, determining the target structure based on the second 2D molecular structure and the second 3D molecular structure comprises: in response to the first evaluation being superior to the second evaluation, training an editing model for predicting an edit operation based on the editing for the first 2D molecular structure; editing the second 2D molecular structure with the trained editing model to determine a third 2D molecular structure; and determining the target structure of the ligand molecule for the target molecule based on the third 2D molecular structure and the second 2D molecular structure.

In some embodiments, determining a first evaluation for a second 3D molecular structure comprising: determining a first normalized value based on the target binding capacity, the first normalized value decreasing as the binding free energy indicated by the target binding capacity increases; determining a second normalized value based on the QED, the second normalized value increasing as the QED increases; determining a third normalized value based on the synthesizability, the third normalized value decreasing as a synthesis difficulty indicated by the synthesizability increases; and determining the first evaluation based on the first, second and third normalized values.

In some embodiments, determining the first evaluation based on the first, second and third normalized values comprises: determining the first evaluation based on a first weight associated with the first normalized value, a second weight associated with the second normalized value and a third weight associated with the third normalized value according to the first, second and third normalized values.

In some embodiments, the first 2D molecular structure is generated by applying a first number of edit operations on an initial 2D molecular structure, and the probability is further based on the first number.

In some embodiments, the first 2D molecular structure is generated by applying a first number of edit operations on an initial 2D molecular structure, and determining a target structure for a ligand molecule of the target molecule comprises: incrementing the first number to determine the second number; and if the second number reaches a predetermined threshold, determining the second 3D molecular structure as the target structure.

In a second aspect of the present disclosure, an apparatus for designing ligand molecules is provided. The apparatus comprises: an editing module configured to edit a first 2D molecular structure to determine a second 2D molecular structure, the editing at least comprising: deleting a 2D structural fragment from the first 2D molecular structure, or adding a 2D structural fragment to the first 2D molecular structure; and a generating module configured to determine a set of candidate 3D molecular structures corresponding to the second 2D molecular structure based on a first 3D molecular structure corresponding to the first 2D molecular structure and the editing and to determine a second 3D molecular structure corresponding to the second 2D molecular structure based on a binding capacity between the set of candidate 3D molecular structures and a target molecule, wherein the editing module is further configured to determine a target structure of a ligand molecule for the target molecule based on the second 3D molecular structure.

In some embodiments, the editing module is further configured to: determine an edit operation to be applied to the first 2D molecular structure with an operation prediction model and based on a feature representation corresponding to the first 2D molecular structure; and edit the first 2D molecular structure based on the determined edit operation.

In some embodiments, the editing module is further configured to: determine a set of probabilities associated with a set of predetermined edit operations with the operation prediction model and based on the feature representation, wherein the set of predetermined edit operations comprise: adding a specific 2D structure fragment at a specific atom in the first 2D molecular structure, or deleting a specific bond from the first 2D molecular structure; and determine the edit operation to be applied to the first 2D molecular structure from the set of predetermined edit operations based on the set of probabilities.

In some embodiments, the editing module is further configured to: select a target 2D structure fragment from a fragment library, the fragment library including a plurality of 2D structure fragments; and add the target 2D structure fragment to a specific atom in the first 2D molecular structure.

In some embodiments, the generating module is further configured to: determine the set of candidate 3D molecular structures based on the editing with the first 3D molecular structure, wherein the set of candidate structures have a partial 3D structure corresponding to the first 3D molecular structure, the partial 3D structure corresponding to a partial 2D structure that is unmodified by the edit operation.

In some embodiments, the editing is adding a target 2D structure fragment to the first 2D molecular structure, and the generating module is further configured to: determine a configuration constraint based on the first 3D molecular structure corresponding to the first 2D molecular structure; generate a plurality of candidate 3D molecular structures corresponding to the editing based on the configuration constraint, configuration constraint specifying an extent to which the first 3D molecular structure is adjusted in process of generating the plurality of candidate 3D molecular structures; and perform an energy optimization on the plurality of candidate 3D molecular structures based on the configuration constraint to determine the set of candidate 3D molecular structures.

In some embodiments, binding is determined based on the binding free energy between a set of candidate 3D structural fragments and the target molecule.

In some embodiments, the editing module is further configured to: determine a first evaluation for the second 3D molecular structure, the first evaluation indicating at least one of the following: a target binding capacity between the second 3D molecular structure and the target molecule, quantitative estimate of drug-likeness (QED) of the second 3D molecular structure, or synthesizability of the second 3D molecular structure; determine a probability of acceptance of the second 2D molecular structure based on the first evaluation and a second evaluation of the first 3D molecular structure; and determine the target structure based on the second 2D molecular structure and the second 3D molecular structure according to the probability.

In some embodiments, the editing module is further configured to: in response to the first evaluation being superior to the second evaluation, train an editing model for predicting an edit operation based on the editing for the first 2D molecular structure; edit the second 2D molecular structure with the trained editing model to determine a third 2D molecular structure; and determine the target structure of the ligand molecule for the target molecule based on the third 2D molecular structure and the second 2D molecular structure.

In some embodiments, the generating module is further configured to: determine a first normalized value based on the target binding capacity, the first normalized value decreasing as the binding free energy indicated by the target binding capacity increases; determine a second normalized value based on the QED, the second normalized value increasing as the QED increases; determine a third normalized value based on the synthesizability, the third normalized value decreasing as a synthesis difficulty indicated by the synthesizability increases; and determine the first evaluation based on the first, second and third normalized values.

In some embodiments, the generating module is further configured to: determine the first evaluation based on a first weight associated with the first normalized value, a second weight associated with the second normalized value and a third weight associated with the third normalized value according to the first, second and third normalized values.

In some embodiments, the first 2D molecular structure is generated by applying a first number of edit operations on an initial 2D molecular structure, and the probability is further based on the first number.

In some embodiments, the first 2D molecular structure is generated by applying a first number of edit operations on an initial 2D molecular structure, and the editing module is further configured to: increment the first number to determine the second number; and if the second number reaches a predetermined threshold, determine the second 3D molecular structure as the target structure.

In a third aspect of the present disclosure, an electronic device is provided, comprising: a memory and a processor; wherein the memory is configured to store one or more computer instructions, wherein the one or more computer instructions, when executed by the processor, implement the method according to the first aspect of the present disclosure.

In a fourth aspect of the present disclosure, there is provided a computer-readable storage medium having one or more computer instructions stored thereon, wherein the one or more computer instructions, when executed by a processor, implement the method according to the first aspect of the present disclosure.

In a fifth aspect of the present disclosure, there is provided a computer program product comprising one or more computer instructions, wherein the one or more computer instructions, when executed by a processor, implement a method according to a first aspect of the present disclosure.

According to various embodiments of the present disclosure, a new 3D molecular structure can be constructed using the 3D molecular structure in the prior state, for evaluating whether the edited 3D molecular structure (or its corresponding 2D molecular structure) can be accepted for determining the target structure of the final ligand molecule. Based on this method, embodiments of the present disclosure can improve the construction efficiency of the 3D molecular structure, especially improve the search for binding configurations between the 3D molecular structure and the target molecule, thereby improving the efficiency of determining ligand molecules.

### BRIEF DESCRIPTION OF THE DRAWINGS

The above and other features, advantages and aspects of the various embodiments of the present disclosure will become more apparent in conjunction with the accompanying drawings and with reference to the following detailed description. In the drawings, identical or similar reference numerals denote identical or similar elements, wherein:

FIG. 1 shows a schematic block diagram of a computing device capable of implementing some embodiments of the present disclosure;

FIG. 2 shows a schematic block diagram of a design module according to some embodiments of the present disclosure;

FIG. 3 shows a schematic diagram of constructing a 3D molecular structure in accordance with some embodiments of the present disclosure;

FIG. 4 shows a schematic view of constructing a 3D molecular structure according to still other embodiments of the present disclosure; and

FIG. 5 shows a flowchart of an example method for designing ligand molecules according to some embodiments of the present disclosure.

### DETAILED DESCRIPTION

The following will describe embodiments of the present disclosure in more detail with reference to the accompanying drawings. Although certain embodiments of the present disclosure are displayed in the drawings, it should be understood that the present disclosure can be implemented in various forms and should not be construed as limited to the embodiments set forth herein. On the contrary, these embodiments are provided for a more thorough and complete understanding of the present disclosure. It should be understood that the accompanying drawings and embodiments of the present disclosure are for illustrative purposes only and are not intended to limit the scope of protection of the present disclosure.

In the description of embodiments of the present disclosure, the term “comprising” and similar terms should be understood as open-ended inclusion, that is, “including but not limited to”. The term “based on” should be understood as “at least partially based on”. The term “one embodiment” or “the embodiment” should be understood as “at least one embodiment”. The terms “first”, “second”, etc. may refer to different or identical objects. The following text may also include other explicit and implicit definitions.

As discussed above, with the development of computer technology, computer-aided technologies such as Machine Learning are gradually being applied to the process of drug molecule discovery. People are also paying more and more attention to the efficiency of drug molecule discovery based on computer-aided technology.

According to embodiments of the present disclosure, a solution for designing ligand molecules is provided. In this scheme, the first 2D molecular structure can be edited to determine the second 2D molecular structure, where editing at least includes: deleting 2D structural fragments from the first 2D molecular structure or adding 2D structural fragments to the first 2D molecular structure. Furthermore, a set of candidate 3D molecular structures corresponding to the second 2D molecular structure can be determined based on the first 3D molecular structure corresponding to the first 2D molecular structure and editing. The second 3D molecular structure corresponding to the second 2D molecular structure can be determined based on the binding capacity between the set of candidate 3D molecular structures and the target molecule. Furthermore, the target structure of the ligand molecule for the target molecule can be determined based on the second 3D molecular structure.

Various embodiments of the present disclosure can utilize the 3D molecular structure of a prior state to construct new 3D molecular structures for evaluating whether they can be used to determine ligand molecules. In this way, embodiments of the present disclosure can improve the construction efficiency of 3D molecular structures, especially the search for binding configurations between 3D molecular structures and target molecules, thereby improving the efficiency of determining ligand molecules.

The following describes the basic principles and several example implementations of the present disclosure with reference to the accompanying drawings.

### Example Device

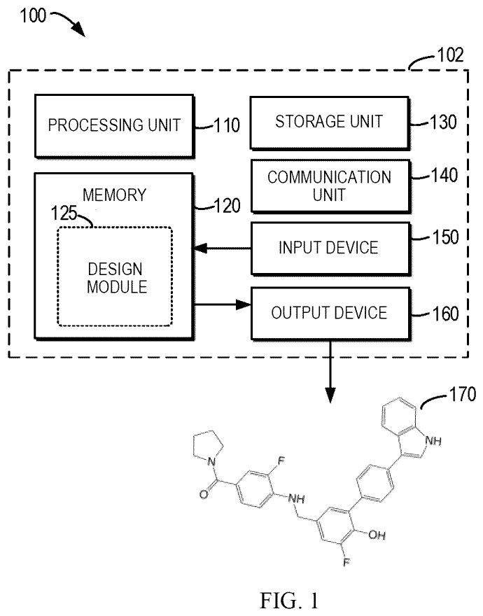

FIG. 1 shows a schematic block diagram of an example device 100 that can be used to implement embodiments of the present disclosure. It should be understood that the device 100 shown in FIG. 1 is merely exemplary and should not constitute any limitation on the functionality and scope of the implementations described in this disclosure. As shown in FIG. 1, the components of the device 100 may include but are not limited to one or more processors or processing units 110, memory 120, storage devices 130, one or more communication units 140, one or more input devices 150, and one or more output devices 160.

In some implementations, device 100 can be implemented as various user end points or service end points. Service end points can be servers, large computing devices, etc. provided by various service providers. User end points are any type of mobile end point, fixed end point, or portable end point, including mobile phones, multimedia computers, multimedia tablets, internet nodes, communicators, desktop computers, laptop computers, notebook computers, netbook computers, tablet computers, personal communication system (PCS) devices, personal navigation devices, personal digital assistants (PDAs), audio/Mobile Pentium 4, digital cameras/camcorders, positioning devices, television receivers, radio broadcast receivers, e-book devices, gaming devices, or any combination thereof, including accessories and peripherals of these devices or any combination thereof. It is also foreseeable that device 100 can support any type of user-specific interface (such as “wearable” circuits, etc.).

The processing unit 110 may be an actual or virtual processor and is capable of performing various processing according to programs stored in the memory 120. In a multiprocessor system, multiple processing units execute computer-executable instructions in parallel to improve the parallel processing capability of the device 100. The processing unit 110 may also be referred to as a central processing unit (CPU), a microprocessor, a controller, or a microcontroller.

Device 100 typically includes multiple computer storage media. Such media can be any available media accessible to device 100, including but not limited to volatile and nonvolatile media, removable and non-removable media. Memory 120 can be volatile memory (such as registers, caches, random access memory (RAM)), nonvolatile memory (such as read-only memory (ROM), electrically erasable programmable read-only memory (EEPROM), flash memory), or some combination thereof. Memory 120 can include one or more design modules 125 that are configured to perform various implemented functions described herein. Design modules 125 can be accessed and operated by processing unit 110 to implement corresponding functions. Storage device 130 can be removable or non-removable media and can include machine-readable media that can be used to store information and/or data and can be accessed within device 100.

The functions of the components of device 100 can be implemented in a single computing cluster or multiple computing machines, which can communicate through communication connections. Therefore, device 100 can operate in a networked environment using logical connections with one or more other servers, personal computers (PCs), or another general network node. Device 100 can also communicate with one or more external devices (not shown) through communication unit 140 as needed. External devices such as database 145, other storage devices, servers, display devices, etc. can communicate with one or more devices that enable users to interact with device 100, or with any device (such as network interface cards, modems, etc.) that enables device 100 to communicate with one or more other computing devices. Such communication can be performed via input/output (I/O) interfaces (not shown).

Input device 150 may be one or more of various input devices, such as a mouse, keyboard, trackball, voice input device, camera, etc. Output device 160 may be one or more output devices, such as a display, speaker, printer, etc.

In some implementations, device 100 may, for example, receive an identification corresponding to a target molecule (e.g., a targeted protein molecule) through input device 150. For example, a user may input a PDB file through input device 150 to indicate the corresponding target molecule.

In some implementations, the design module 125 may use the editing model to iteratively edit the molecular structure to determine the target structure of the final ligand molecule 170. The process of determining the target structure of the ligand molecule 170 will be described in detail below.

It should be appreciated that although the ligand molecule 170 output in FIG. 1 is shown as a 2D molecular structure. In some embodiments, the output device 160 may output, for example, a 3D molecular structure.

### Ligand Molecular Design

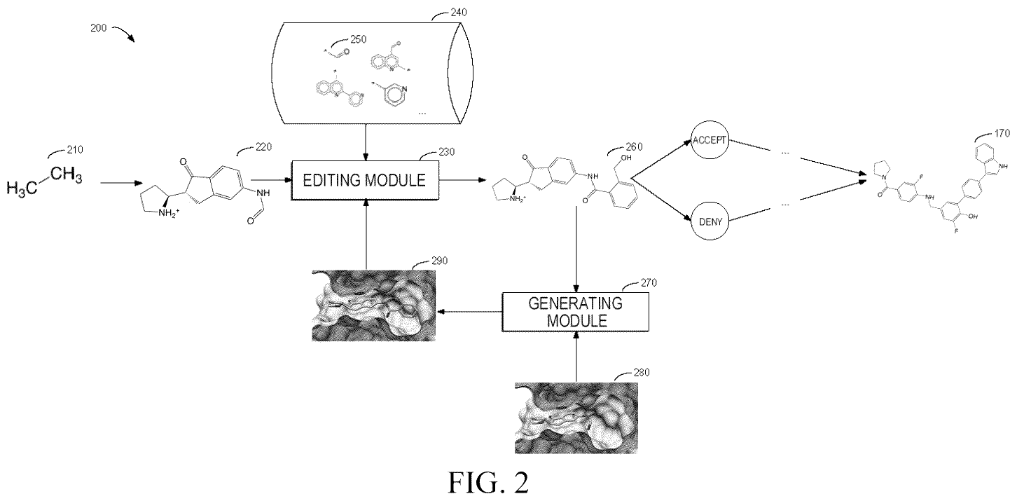

Referring to FIG. 2, a block diagram of a design module 125 according to some embodiments of the present disclosure is shown. As shown in FIG. 2, the design module 125 includes a plurality of modules for implementing the process of designing ligand molecules according to some embodiments of the present disclosure. The design module 125 includes an editing module 230 and a generating module 240.

In some embodiments, the editing module 230 can edit the first 2D molecular structure 220. Specifically, the edit may include deleting a 2D structural fragment from the first 2D molecular structure 220, and such edit can be referred to as a “delete edit operation.” Alternatively, the edit may also include adding a new 2D structural fragment to the first 2D molecular structure 220, and such edit can be referred to as an “add edit operation.”

For the “delete edit operation”, the editing module 230 may determine the bond to be deleted from the first 2D molecular structure 220 and accordingly delete the 2D structure fragment associated with the bond to be deleted from the first molecular structure. For example, the editing module 230 may delete the group associated with the bond to be deleted from the first 2D molecular structure 220.

For the “add edit operation”, the editing module 230 can determine the atoms to be edited in the first 2D molecular structure 220 and accordingly select a 2D structural fragment from the fragment library 240 to attach to the first 2D molecular structure 220. During the process of “add edit operation”, the atoms to be edited in the first 2D molecular structure 220 can add new bonds to the selected 2D fragment to construct a new molecular structure.

In some embodiments, the fragment library 240 may include a plurality of 2D structural fragments 250. In some embodiments, the plurality of 2D structural fragments 250 may be determined, for example, based on experimental knowledge. Alternatively, the plurality of 2D structural fragments 250 may also be constructed based on existing drug molecules.

In some embodiments, the first 2D molecular structure 220 can be obtained, for example, from an initial 2D molecular structure 210 (e.g., ethane molecule C₂H₆ shown in FIG. 2) after at least one edit procedure as discussed above. Alternatively, the first 2D molecular structure 220 can also be the initial 2D molecular structure. Accordingly, as the initial 2D molecular structure, it can be selected by the editing module 230 or determined by the editing module 230 according to inputs. As shown in FIG. 2, the editing module 230 can use the deployed editing model to edit the first 2D molecular structure 220 to obtain the second 2D molecular structure 260. The editing model can be implemented based on a machine learning model. Specific details about the editing module 230 and the editing model will be described in detail below.

As shown in FIG. 2, the design module 125 may further include a generating module 270. In some embodiments, the generating module 270 may be used to determine a 3D molecular structure corresponding to the second 2D molecular structure 260.

In some embodiments, the generating module 270 can efficiently construct the second 3D molecular structure 290 corresponding to the second 2D molecular structure 260, for example, based on the first 3D molecular structure 280 corresponding to the first 2D molecular structure 220 and an edit operation performed by the editing module 230 on the first 2D molecular structure 220.

In some embodiments, the editing module 230 and/or the production module 270 can also determine an evaluation (also referred to as a first evaluation for convenience of description) for the second 3D molecular structure 290. For example, the editing module 230 may determine a first evaluation based on the binding capacity between the second 3D molecular structure 290 and the target molecule 170. Additionally, the generating module 270 may also determine a first evaluation based on factors such as Quantitative Estimate of Drug-likeness (QED) and/or synthesizability.

Further, the editing module 230 may further determine whether the second 2D molecular structure 260 is acceptable based on a first evaluation for the second 3D molecular structure 290 and a second evaluation for the first 3D molecular structure 280. If the second 2D molecular structure 260 is determined to be acceptable, it may be determined as the next state of a Markov chain to iteratively determine the target structure of the final ligand molecule 170, for example.

On the contrary, if the second 2D molecular structure 260 is determined to be rejected based on the first evaluation and the second evaluation, the editing module 230 may discard the second 2D molecular structure and continue to determine a new edit based on the first 2D molecular structure 220, thereby iteratively determining the target structure of the final ligand molecule 170.

It should be understood that the editing module 230 may determine a second evaluation with respect to the first 3D molecular structure 280 based on a similar procedure. In some embodiments, if the first evaluation is superior to the second evaluation, the editing module 230 may further train the editing model deployed in the editing module 230 based on the edit operation performed for the first 2D molecular structure 220.

In some embodiments, the editing module 230 may iteratively perform editing with the trained editing model based on the second 2D molecular structure 260 until the target structure for the ligand molecule 170 of the target molecule is determined.

In some embodiments, for example, the editing module 230 may terminate the iteration after performing a predetermined number of edits on the initial 2D molecular structure 210 and determine the final output 2D molecular structure as the target structure of the ligand molecule 170. Alternatively, the editing module 230 can determine the 3D molecular structure corresponding to the final 2D molecular structure as the target structure of the ligand molecule 170.

In some embodiments, the editing module 230 may also determine whether it converges based on the degree of change in the evaluated molecular structure after each iteration of editing. For example, if the change in the evaluated molecular structure after a predetermined number of iterations is less than a predetermined threshold, the editing module 230 may determine that it has converged and determine the final output molecular structure as the target structure of the ligand molecule.

The detailed process of self-supervised training will be described in detail below.

### Molecular Structure Editing

As discussed with reference to FIG. 2, the editing module 230 is configured to edit the first 2D molecular structure 220 with the deployed editing model. In some embodiments, the editing model may be implemented, for example, based on an appropriate machine learning model.

Specifically, the editing module 230 may first determine a feature representation of the first 2D molecular structure 220. In some embodiments, the first 2D molecular structure 220 may be represented as a graph x, which may have n atoms and n bonds, for example. In some embodiments, the editing module 230 may represent the first 2D molecular structure 220 as:

$${h}_{α}^{node} = {{ℳ}_{θ} ( x )}_{a} ∈ {ℝ}^{d} \qquad ( 1 )$$

$${h}_{b}^{edge} = Concat ( {h}_{w}^{node} , {h}_{v}^{node )} ) ∈ {ℝ}^{2 d} \qquad ( 2 )$$

where a represents an index of the atom in the first 2D molecular structure 220, h_{α}^{node} represents the hidden layer feature corresponding to the atom; w and ν represent the atoms connected by the bond b in the first 2D molecular structure 220, its corresponding hidden layer feature is represented by h_{b}^{edge}, [symbol] _{θ}(·) represents MPNN (Message Passing Neural Network) with the model parameter θ.

Furthermore, the editing module 230 may utilize an operation prediction model and determine a set of probabilities associated with a set of predetermined edit operations based on the feature representations determined according to the formulas (1) and/or (2). Such predetermined edit operations include, for example, adding a specific 2D structure fragment at a specific atom in the first 2D molecular structure 220, or deleting a specific bond from the first 2D molecular structure 220.

Such a process can be expressed as:

$${p}_{add} ( {a}_{u} \vert x ) = {σ ( {\{ {MLP}_{node} ( {h}_{u}^{node} ) \}}_{u = 1}^{n} )}_{u} \qquad ( 3 )$$

$${p}_{frag} ( {f}_{k} \vert x , {a}_{u} ) = {σ ( {MLP}_{node}^{′} ( {h}_{u}^{node} ) )}_{k} \qquad ( 4 )$$

$${p}_{del} ( {e}_{b} \vert x ) = {σ ( {\{ {MLP}_{edge} ( {h}_{b}^{edge} ) \}}_{b = 1}^{2 m} )}_{b} \qquad ( 5 )$$

where MLP_{node}(·)∈ [symbol] , MLP′_{node}(·)∈ [symbol] , MLP_{edge}(·)∈R [symbol] represents an independent multi-layer perceptron (MLP) and σ(·) represents the Softmax operation.

Further, the editing module 230 may determine the probability corresponding to different predetermined edit operations as follows:

$${p}_{c} ( add \vert x ) = {p}_{c} ( del \vert x ) = \frac{1}{2} \qquad ( 6 )$$

$$q ( {x}_{( u , k )}^{′} \vert x ) = {p}_{c} ( add \vert x ) \cdot {p}_{add} ( u \vert x ) \cdot {p}_{frag} ( k \vert x , u ) \qquad ( 7 )$$

$$q ( {x}_{( b )}^{′} \vert x ) = {p}_{c} ( del \vert x ) \cdot {p}_{del} ( b \vert x ) \qquad ( 8 )$$

where x′_{(u,k)} represents the molecule obtained by adding the k-th 2D structure fragment in the fragment library 240 to the atom x′_{(b)} represents the molecule obtained by deleting the bond b and the attached fragment from the first 2D molecular structure 220.

Further, the editing module 230 may determine an edit operation to be applied to the first 2D molecular structure 220 from a set of predetermined edit operations based on the determined set of probabilities. For example, the editing module 230 can sample to determine the edit operation to be applied based on this set of probabilities.

### 3D Molecular Structure Generation

In some embodiments, as discussed above with reference to FIG. 2, the generating module 270 may construct a second 3D molecular structure 290 for the second 2D molecular structure 260 based on the first 3D molecular structure 220 corresponding to the first 2D molecular structure 280.

In some embodiments, the generating module 270 may determine a set of candidate 3D molecular structures based on the edit applied to the first 2D molecular structure 220 and utilize the first 3D molecular structure 280, where the set of candidate 3D molecular structures have partial 3D structure corresponding to a first 3D molecular structure 280, and the partial 3D structure is corresponding to a partial 2D structure that is unmodified by the editing.

In this way, the generating module 270 may perform constrained 3D molecular structure construction based on the first 3D molecular structure 280, thereby determining the second 3D molecular structure 290 more efficiently.

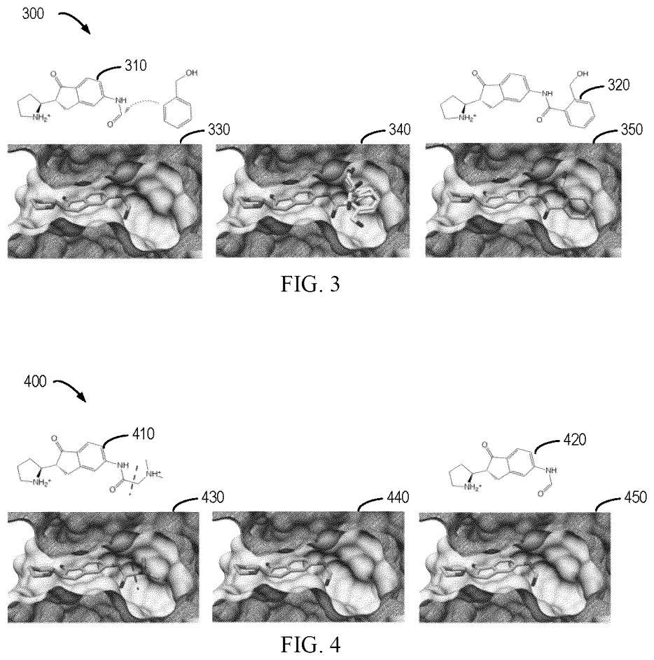

FIG. 3 shows a schematic diagram 300 of constructing a 3D molecular structure according to some embodiments of the present disclosure. As shown in FIG. 3, for the add edit operation of adding the target 2D structure fragment, unlike the traditional generation process, the generating module 270 can consider the first 3D molecular structure during the generation process, that is, introduce configuration constraints corresponding to the first 3D molecular structure.

Specifically, the generating module 270 may determine a configuration constraint(s) based on the first 3D molecular structure. The constraint is used to limit the extent to which the first 3D molecular structure is adjusted in subsequent generation processes. By way of example, the generating module 270 may determine a constraint(s) related to atomic distances based on the first 3D molecular structure (e.g., 3D molecular structure 330 in FIG. 3, which corresponds to 2D molecular structure 310).

Furthermore, the generating module 270 can generate multiple candidate 3D molecular structures based on the configuration constraint. By way of example, the generating module 270 can use appropriate configuration generation tools to generate multiple candidate 3D molecular structures under configuration constraint.

Additionally, the generating module 270 may further perform energy optimization on a plurality of candidate 3D molecular structures based on configuration constraint, thereby determining a set of candidate 3D molecular structures (e.g., candidate 3D molecular structure 340 in FIG. 3).

Furthermore, the generating module 270 may determine the second 3D molecular structure 290 corresponding to the second 2D molecular structure 260 based on the binding capacity between the set of candidate 3D molecular structures and the target molecule. Specifically, the generating module 270 may determine a target 3D molecular structure from the set of candidate 3D molecular structures which has the minimum binding free energy with the target molecule as the second 3D molecular structure corresponding to the second 2D molecular structure (e.g., the 2D molecular structure 320 in FIG. 3, which is determined by performing an add edit operation on the 2D molecular structure 310).

FIG. 4 shows a schematic diagram of constructing a 3D molecular structure according to some further embodiments of the present disclosure. As shown in FIG. 4, for a delete edit operation to delete a target 2D structure fragment, the generating module 270 may retain a portion of the first 3D molecular structure (e.g., the 3D molecular structure 430 in FIG. 4, which corresponds to the 2D molecular structure 410) which is not deleted by the delete edit operation.

Further, the generative model 270 may release the retained portion of the 3D molecular structure and perform local energy optimization to determine candidate 3D molecular structures (e.g., 3D molecular structure 440 in FIG. 4).

Furthermore, the generating module 270 may determine the second 3D molecular structure 290 corresponding to the second 2D molecular structure 260 based on the binding capacity between the candidate 3D molecular structure and the target molecule. Specifically, the generating module 270 may minimize the binding free energy with the target molecule to determine the target 3D molecular structure based on the candidate 3D molecular structure as the second 3D molecular structure corresponding to the second 2D molecular structure (e.g., the 2D molecular structure 420 in FIG. 4, which is determined by performing a delete edit operation on the 2D molecular structure 410).

Through the constrained 3D molecular structure construction process, embodiments of the present disclosure can greatly reduce the computational overhead required for constructing 3D molecular structures, thereby improving the efficiency of constructing 3D molecular structures. In addition, in considering the process of minimizing the binding energy with the target molecule, the constrained 3D molecular structure construction process can greatly improve the computational efficiency of searching for the minimum binding energy.

### Self-Supervised Training

In some embodiments, as discussed above with reference to FIG. 2, the editing module 230 may also be applied to the first 2D molecular structure 220 based on the edit operation from supervised training editing model.

As discussed above, the edit operation applied to the first 2D molecular structure 220 is determined based on the sampling of probabilities. In some embodiments, the design module 125 may perform a plurality of sampling in parallel, for example, to obtain multiple candidate 2D molecular structures based on the first 2D molecular structure 220.

In some embodiments, the editing module 230 may determine an evaluation for each of the candidate 2D molecular structures. As discussed above, for example, the evaluation may be based on the binding capacity between the 3D molecular structure corresponding to the candidate 2D molecular structure and the target molecule, the QED of the 3D molecular structure, and/or the synthesizability of the 3D molecular structure.

In this way, embodiments of the present disclosure can simultaneously generate multiple target ligand molecules.

In some embodiments, the editing module 230 may normalize the binding capacity, drug-likeness and synthesizability. For binding capacity, the editing module 230 may determine the binding free energy between the molecular structure and the target molecule. By way of example, it may be generated by molecular docking application. Further, the editing module 230 may determine a first normalized value based on the binding capacity and wherein the first normalized value decreases as the binding free energy indicated by the target binding capacity increases. By way of example, the first normalized value may be expressed as:

$${s}_{D} ( x ) = {e}^{- D ( x )} \qquad ( 9 )$$

For EQD, the editing module 230 can determine a second normalized value that increases as the EQD increases. For example, the second normalized value can be represented as:

$${s}_{QED} ( x ) = QED ( x ) \qquad ( 10 )$$

where QED(·) represents the QED score which can be calculated by RDKit, for example.

For synthesizability, the editing module 230 can determine a third normalized value which decreases as the synthesis difficulty indicated by synthesizability increases. By way of example, the third normalized value can be represented as:

$${s}_{SA} ( x ) = ( 10 - SA ( x ) ) / 9 \qquad ( 11 )$$

where S_{SA}(x) represents a synthesis difficulty score.

Further, the editing module 230 may determine a first evaluation based on the first, second, and third normalized values.

By way of example, the first evaluation can be expressed as:

$$π ( x ) = {[ {s}_{QED}^{{w}_{1}} ( x ) \times {s}_{SA}^{{w}_{2}} ( x ) \times {s}_{D}^{{w}_{3}} ( x ) ]}^{1 / ( {w}_{1} + {w}_{2} + {w}_{3} )} \qquad ( 12 )$$

where w₁, w₂ and w₃ represent the weight corresponding to QED, the weight corresponding to the synthesizability and the weight corresponding to binding capacity, respectively.

In some embodiments, the editing module 230 may determine a probability that the second 2D molecular structure 260 is accepted based on a first evaluation and a second evaluation for the first 2D molecular structure 220. This probability may be expressed, for example:

$$𝒜 ( x , {x}^{′} ) = min \{ 1 , \frac{{π}^{α} ( {x}^{′} ) q ( x \vert {x}^{′} )}{{π}^{α} ( x ) q ( {x}^{′} \vert x )} \} \qquad ( 13 )$$

where π^{α}(x′) represents the first evaluation for the second 2D molecular structure 260, π^{α}(x) represents the second evaluation for the first 2D molecular structure 220,

$$α = \frac{1}{T}$$

where T′ represents the temperature coefficient which is determined based on the annealing mechanism. In some embodiments, the temperature coefficient T is determined based on the number of edit operations that have been applied to the first 2D molecular structure. By way of example, if the first 2D molecular structure is generated by applying a first number of edit operations on the initial 2D molecular structure, the temperature coefficient T is associated with the first number.

In some embodiments, the design module 125 may determine the probability of acceptance or rejection of the second 2D molecular structure 260 based on Equation (13). As discussed with reference to FIG. 2, if the second 2D molecular structure 260 is accepted, the involving module 125 may further iteratively edit based on the second 2D molecular structure 260 to determine the target structure of the ligand molecule 170. On the contrary, if the second 2D molecular structure is rejected, the design module 125 may further iteratively edit based on the first 2D molecular structure 220 to determine the target structure of the ligand molecule 170.

In this way, some editing operations that lead to reduced evaluation can also be randomly retained, thereby increasing the diversity of drug molecule generation.

In some embodiments, for a candidate 2D molecular structure with an evaluation superior to the first 2D molecular structure 220, the editing module 230 may further train the editing model based on an edit operation corresponding to the generation of the candidate 2D molecular structure. In some embodiments, editing model can be trained based on maximum likelihood estimation (MLE).

In some embodiments, for example, the editing module 230 may terminate the iteration after a predetermined number of edits have been made to the initial 2D molecular structure 210, and the final output of the 2D molecular structure is determined as the target structure of ligand molecule 170.

If the predetermined number of edits has not been performed, the editing module 230 can generate a new third 2D molecular structure based on the second 2D molecular structure with use the retrained editing model, and iteratively execute accordingly. During the iteration process, the editing module 230 can increment the number of edits that have been made until the predetermined number of edits is reached before exiting the iteration.

On the contrary, if a predetermined number of edits have been made for generating the second 2D molecular structure 260 (e.g., the number reaches a predetermined threshold), the editing module 230 may determine the second 3D molecular structure 290 and/or the second 2D molecular structure 260 as the target structure.

In some embodiments, the editing module 230 may also determine whether it converges based on the extent of change in the evaluated molecular structure after each iteration of editing. For example, if the change in the evaluated molecular structure after a predetermined number of iterations is less than a predetermined threshold, the editing module 230 may determine that it has converged and determine the final output molecular structure as the target structure of the ligand molecule.

### Example Process

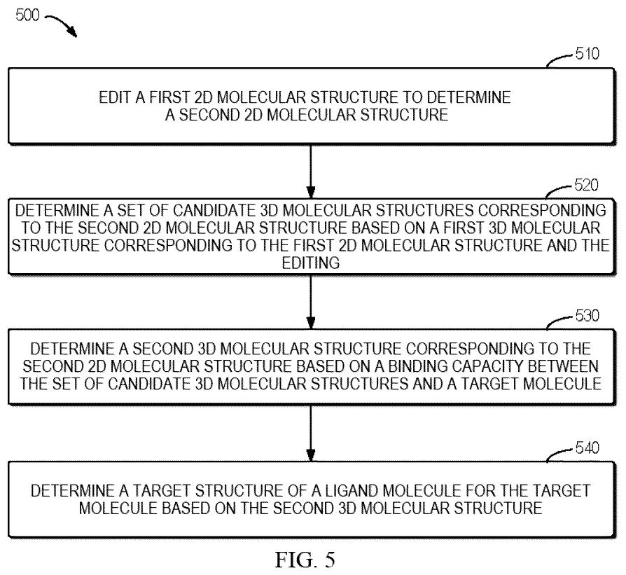

FIG. 5 shows a flowchart of a method 500 for designing ligand molecules in accordance with some embodiments of the present disclosure. Method 500 may be implemented by computing device 100, for example, at design module 125 in memory 120 of computing device 100.

As shown in FIG. 5, at block 510, the computing device 100 edits a first 2D molecular structure to determine a second 2D molecular structure, editing comprising at least: deleting a 2D structure fragment from the first 2D molecular structure or adding a 2D structure fragment to the first 2D molecular structure.

At block 520, the computing device 100 determines a set of candidate 3D molecular structures corresponding to the second 2D molecular structure based on a first 3D molecular structure corresponding to the first 2D molecular structure and the editing.

At block 530, the computing device 100 determines a second 3D molecular structure corresponding to the second 2D molecular structure based on a binding capacity between the set of candidate 3D molecular structures and a target molecule.

At block 540, the computing device 100 determines the target structure of the ligand molecule for the target molecule based on the second 3D molecular structure.

The following are some example embodiments of the present disclosure.

In some embodiments, editing the first 2D molecular structure comprises: determining an edit operation to be applied to the first 2D molecular structure with an operation prediction model and based on a feature representation corresponding to the first 2D molecular structure; and editing the first 2D molecular structure based on the determined edit operation.

In some embodiments, determining the edit operation to be applied to the first 2D molecular structure comprises: determining a set of probabilities associated with a set of predetermined edit operations with the operation prediction model and based on the feature representation, wherein the set of predetermined edit operations comprise: adding a specific 2D structure fragment at a specific atom in the first 2D molecular structure, or deleting a specific bond from the first 2D molecular structure; and determining the edit operation to be applied to the first 2D molecular structure from the set of predetermined edit operations based on the set of probabilities.

In some embodiments, adding a 2D structure fragment comprises: selecting a target 2D structure fragment from a fragment library, the fragment library including a plurality of 2D structure fragments; and adding the target 2D structure fragment to a specific atom in the first 2D molecular structure.

In some embodiments, determining a set of candidate 3D molecular structures corresponding to the second 2D molecular structure comprises: determining the set of candidate 3D molecular structures based on the editing with the first 3D molecular structure, wherein the set of candidate structures have a partial 3D structure corresponding to the first 3D molecular structure, the partial 3D structure corresponding to a partial 2D structure that is unmodified by the editing.

In some embodiments, the editing is adding a target 2D structure fragment to the first 2D molecular structure, and determining the set of candidate 3D molecular structures comprises: determining a configuration constraint based on the first 3D molecular structure corresponding to the first 2D molecular structure; generating a plurality of candidate 3D molecular structures corresponding to the editing based on the configuration constraint, configuration constraint specifying an extent to which the first 3D molecular structure is adjusted in process of generating the plurality of candidate 3D molecular structures; and performing an energy optimization on the plurality of candidate 3D molecular structures based on the configuration constraint to determine the set of candidate 3D molecular structures.

In some embodiments, the binding is determined based on a binding free energy between the set of candidate 3D structure fragments and the target molecule.

In some embodiments, determining the target structure of a ligand molecule for the target molecule comprises: determining a first evaluation for the second 3D molecular structure, the first evaluation indicating at least one of the following: a target binding capacity between the second 3D molecular structure and the target molecule, quantitative estimate of drug-likeness (QED) of the second 3D molecular structure, or synthesizability of the second 3D molecular structure; determining a probability of acceptance of the second 2D molecular structure based on the first evaluation and a second evaluation of the first 3D molecular structure; and determining the target structure based on the second 2D molecular structure and the second 3D molecular structure according to the probability.

In some embodiments, determining the target structure based on the second 2D molecular structure and the second 3D molecular structure comprises: in response to the first evaluation being superior to the second evaluation, training an editing model for predicting an edit operation based on the editing for the first 2D molecular structure; editing the second 2D molecular structure with the trained editing model to determine a third 2D molecular structure; and determining the target structure of the ligand molecule for the target molecule based on the third 2D molecular structure and the second 2D molecular structure.

In some embodiments, determining a first evaluation for a second 3D molecular structure comprising: determining a first normalized value based on the target binding capacity, the first normalized value decreasing as the binding free energy indicated by the target binding capacity increases; determining a second normalized value based on the QED, the second normalized value increasing as the QED increases; determining a third normalized value based on the synthesizability, the third normalized value decreasing as a synthesis difficulty indicated by the synthesizability increases; and determining the first evaluation based on the first, second and third normalized values.

In some embodiments, determining the first evaluation based on the first, second and third normalized values comprises: determining the first evaluation based on a first weight associated with the first normalized value, a second weight associated with the second normalized value and a third weight associated with the third normalized value according to the first, second and third normalized values.

In some embodiments, the first 2D molecular structure is generated by applying a first number of edit operations on an initial 2D molecular structure, and the probability is further based on the first number.

In some embodiments, the first 2D molecular structure is generated by applying a first number of edit operations on an initial 2D molecular structure, and determining a target structure for a ligand molecule of the target molecule comprises: incrementing the first number to determine the second number; and if the second number reaches a predetermined threshold, determining the second 3D molecular structure as the target structure.

The functions described above in this article can be at least partially performed by one or more hardware logic components. For example, without limitation, exemplary types of hardware logic components that can be used include: field programmable gate arrays (FPGAs), application specific integrated circuits (ASICs), application specific standard products (ASSPs), system-on-chip (SOCs), load programmable logic devices (CPLDs), and so on.

The program code for implementing the method of the present disclosure may be written in any combination of one or more programming languages. The program code may be provided to a processor or controller of a general-purpose computer, special-purpose computer, or other programmable data processing device, such that the program code, when executed by the processor or controller, causes the functions/operations specified in the flowchart and/or block diagram to be implemented. The program code may be executed entirely on the machine, partially on the machine, partially as a standalone software package, and partially on a remote machine or entirely on a remote machine or server.

In the context of this disclosure, a machine-readable medium can be a tangible medium that can contain or store programs for use by or in conjunction with an instruction execution system, device, or device. A machine-readable medium can be a machine-readable signal medium or a machine-readable storage medium. Machine-readable media can include, but are not limited to, electronic, magnetic, optical, electromagnetic, infrared, or semiconductor systems, devices, or devices, or any suitable combination thereof. More specific examples of machine-readable storage media may include electrical connections based on one or more wires, portable computer disks, hard disks, random access memory (RAM), read-only memory (ROM), erasable programmable read-only memory (EPROM or flash memory), optical fibers, portable compact disc read-only memory (CD-ROM), optical storage devices, magnetic storage devices, or any suitable combination thereof.

In addition, although the operations are depicted in a specific order, it should be understood that such operations are required to be performed in the specific order shown or in a sequential order, or that all illustrated operations should be performed to achieve the desired result. Under certain circumstances, multitasking and parallel processing may be advantageous. Similarly, although several specific implementation details are included in the above discussion, these should not be construed as limiting the scope of the present disclosure. Certain features described in the context of individual implementations can also be implemented in combination in a single implementation. Conversely, various features described in the context of a single implementation can also be implemented separately or in any suitable sub-combination in multiple implementations.

Although the subject matter has been described in language specific to structural features and/or methodological logical acts, it should be understood that the subject matter defined in the appended claims is not necessarily limited to the particular features or acts described above. Rather, the particular features and acts described above are merely exemplary forms of implementation of the claims.

1. A method of designing ligand molecules, comprising:
editing a first 2D molecular structure to determine a second 2D molecular structure, the editing at least comprising: deleting a 2D structural fragment from the first 2D molecular structure, or adding a 2D structural fragment to the first 2D molecular structure;
determining a set of candidate 3D molecular structures corresponding to the second 2D molecular structure based on a first 3D molecular structure corresponding to the first 2D molecular structure and the editing;
determining a second 3D molecular structure corresponding to the second 2D molecular structure based on a binding capacity between the set of candidate 3D molecular structures and a target molecule; and
determining a target structure of a ligand molecule for the target molecule based on the second 3D molecular structure.

2. The method of claim 1, wherein editing the first 2D molecular structure comprises:
determining an edit operation to be applied to the first 2D molecular structure with an operation prediction model and based on a feature representation corresponding to the first 2D molecular structure; and
editing the first 2D molecular structure based on the determined edit operation.

3. The method of claim 2, wherein determining an edit operation to be applied to the first 2D molecular structure comprises:
determining a set of probabilities associated with a set of predetermined edit operations with the operation prediction model and based on the feature representation, wherein the set of predetermined edit operations comprise: adding a specific 2D structure fragment at a specific atom in the first 2D molecular structure, or deleting a specific bond from the first 2D molecular structure; and
determining the edit operation to be applied to the first 2D molecular structure from the set of predetermined edit operations based on the set of probabilities.

4. The method of claim 1, wherein adding the 2D structure fragment comprises:
selecting a target 2D structure fragment from a fragment library, the fragment library including a plurality of 2D structure fragments; and
adding the target 2D structure fragment to a specific atom in the first 2D molecular structure.

5. The method of claim 1, wherein determining a set of candidate 3D molecular structures corresponding to the second 2D molecular structure comprises:
determining the set of candidate 3D molecular structures with the first 3D molecular structure based on the editing, wherein the set of candidate structures have a partial 3D structure corresponding to the first 3D molecular structure, the partial 3D structure corresponding to a partial 2D structure that is unmodified by the edit operation.

6. The method of claim 5, wherein the editing is adding a target 2D structure fragment to the first 2D molecular structure, and determining the set of candidate 3D molecular structures comprises:
determining a configuration constraint based on the first 3D molecular structure corresponding to the first 2D molecular structure;
generating a plurality of candidate 3D molecular structures corresponding to the editing based on the configuration constraint, configuration constraint specifying an extent to which the first 3D molecular structure is adjusted in process of generating the plurality of candidate 3D molecular structures; and
performing an energy optimization on the plurality of candidate 3D molecular structures based on the configuration constraint to determine the set of candidate 3D molecular structures.

7. The method of claim 1, wherein the binding is determined based on a binding free energy between the set of candidate 3D structure fragments and the target molecule.

8. The method of claim 1, wherein determining a target structure of a ligand molecule for the target molecule comprises:
determining a first evaluation for the second 3D molecular structure, the first evaluation indicating at least one of the following: a target binding capacity between the second 3D molecular structure and the target molecule, quantitative estimate of drug-likeness (QED) of the second 3D molecular structure, or synthesizability of the second 3D molecular structure;
determining a probability of acceptance of the second 2D molecular structure based on the first evaluation and a second evaluation of the first 3D molecular structure; and
determining the target structure based on the second 2D molecular structure and the second 3D molecular structure according to the probability.

9. The method of claim 8, wherein determining the target structure based on the second 2D molecular structure and the second 3D molecular structure comprises:
in response to the first evaluation being superior to the second evaluation, training an editing model for predicting an edit operation based on the editing for the first 2D molecular structure;
editing the second 2D molecular structure with the trained editing model to determine a third 2D molecular structure; and
determining the target structure of the ligand molecule for the target molecule based on the third 2D molecular structure and the second 2D molecular structure.

10. The method of claim 8, wherein determining a first evaluation for the second 3D molecular structure comprises:
determining a first normalized value based on the target binding capacity, the first normalized value decreasing as the binding free energy indicated by the target binding capacity increases;
determining a second normalized value based on the QED, the second normalized value increasing as the QED increases;
determining a third normalized value based on the synthesizability, the third normalized value decreasing as a synthesis difficulty indicated by the synthesizability increases; and
determining the first evaluation based on the first, second and third normalized values.

11. The method of claim 10, wherein determining the first evaluation based on the first, second and third normalized values comprises:
determining the first evaluation based on a first weight associated with the first normalized value, a second weight associated with the second normalized value and a third weight associated with the third normalized value according to the first, second and third normalized values.

12. The method of claim 8, wherein the first 2D molecular structure is generated by applying a first number of edit operations on an initial 2D molecular structure, and the probability is further based on the first number.

13. The method of claim 1, wherein the first 2D molecular structure is generated by applying a first number of edit operations on an initial 2D molecular structure, and determining a target structure for a ligand molecule of the target molecule comprises:
incrementing the first number to determine the second number; and
if the second number reaches a predetermined threshold, determining the second 3D molecular structure as the target structure.

14. (canceled)

15. An electronic device comprising:
a processor; and
a memory configured to store one or more computer instructions, the one or more computer instructions, when executed by the processor, implementing the method comprising:
editing a first 2D molecular structure to determine a second 2D molecular structure, the editing at least comprising: deleting a 2D structural fragment from the first 2D molecular structure, or adding a 2D structural fragment to the first 2D molecular structure;
determining a set of candidate 3D molecular structures corresponding to the second 2D molecular structure based on a first 3D molecular structure corresponding to the first 2D molecular structure and the editing;
determining a second 3D molecular structure corresponding to the second 2D molecular structure based on a binding capacity between the set of candidate 3D molecular structures and a target molecule; and
determining a target structure of a ligand molecule for the target molecule based on the second 3D molecular structure.

16. A non-transitory computer-readable storage medium having one or more computer instructions stored thereon, the one or more computer instructions, when executed by a processor, implementing the method comprising:
editing a first 2D molecular structure to determine a second 2D molecular structure, the editing at least comprising: deleting a 2D structural fragment from the first 2D molecular structure, or adding a 2D structural fragment to the first 2D molecular structure;
determining a set of candidate 3D molecular structures corresponding to the second 2D molecular structure based on a first 3D molecular structure corresponding to the first 2D molecular structure and the editing;
determining a second 3D molecular structure corresponding to the second 2D molecular structure based on a binding capacity between the set of candidate 3D molecular structures and a target molecule; and
determining a target structure of a ligand molecule for the target molecule based on the second 3D molecular structure.

17. (canceled)

18. The electronic device of claim 15, wherein editing the first 2D molecular structure comprises:
determining an edit operation to be applied to the first 2D molecular structure with an operation prediction model and based on a feature representation corresponding to the first 2D molecular structure; and
editing the first 2D molecular structure based on the determined edit operation.

19. The electronic device of claim 15, wherein determining an edit operation to be applied to the first 2D molecular structure comprises:
determining a set of probabilities associated with a set of predetermined edit operations with the operation prediction model and based on the feature representation, wherein the set of predetermined edit operations comprise: adding a specific 2D structure fragment at a specific atom in the first 2D molecular structure, or deleting a specific bond from the first 2D molecular structure; and
determining the edit operation to be applied to the first 2D molecular structure from the set of predetermined edit operations based on the set of probabilities.

20. The electronic device of claim 15, wherein adding the 2D structure fragment comprises:
selecting a target 2D structure fragment from a fragment library, the fragment library including a plurality of 2D structure fragments; and
adding the target 2D structure fragment to a specific atom in the first 2D molecular structure.

21. The electronic device of claim 15, wherein determining a set of candidate 3D molecular structures corresponding to the second 2D molecular structure comprises:
determining the set of candidate 3D molecular structures with the first 3D molecular structure based on the editing, wherein the set of candidate structures have a partial 3D structure corresponding to the first 3D molecular structure, the partial 3D structure corresponding to a partial 2D structure that is unmodified by the edit operation.

22. The electronic device of claim 18, wherein the editing is adding a target 2D structure fragment to the first 2D molecular structure, and determining the set of candidate 3D molecular structures comprises:
determining a configuration constraint based on the first 3D molecular structure corresponding to the first 2D molecular structure;
generating a plurality of candidate 3D molecular structures corresponding to the editing based on the configuration constraint, configuration constraint specifying an extent to which the first 3D molecular structure is adjusted in process of generating the plurality of candidate 3D molecular structures; and
performing an energy optimization on the plurality of candidate 3D molecular structures based on the configuration constraint to determine the set of candidate 3D molecular structures.
